(19) United States
(12) Patent Application Publication
(10) Pub. No.: US 2009/0105745 A1
(43) Pub. Date: Apr. 23, 2009

(54) Tissue Dilation Systems and Related Methods

(75) Inventor: Brad Culbert, Rancho Santa Margarita, CA (US)

Correspondence Address: KNOBBE MARTENS OLSON & BEAR LLP, 2040 MAIN STREET, FOURTEENTH FLOOR, IRVINE, CA, 92614, US

(73) Assignee: Triage Medical, Irvine, CA (US)

(21) Appl. No.: 11/887,720

(22) PCT Filed: Apr. 4, 2006
(86) PCT No.: PCT/US06/12728

§ 371 Date: Oct. 1, 2007

Related U.S. Application Data
(60) Provisional application No. 60/668,750, filed on Apr. 5, 2005.

(51) Int. Cl. A61M 29/00; A61B 17/32; A61N 1/30

(52) U.S. Cl. 606/192; 606/170; 604/19

(57) ABSTRACT

A tissue dilation system is described which effectively provides an access path from a skin surface region to a target structure located in the body of a patient. The present tissue dilation system includes a tissue penetrating member and a sleeve. The tissue penetrating member includes an expandable body portion that can be inserted through the skin of a patient in a first configuration, and can be deployed into a fully expanded second configuration to dilate tissue surrounding the body portion. The sleeve is placed over the expanded body portion in the fully expanded second configuration. When the tissue penetrating member is removed from the sleeve, an access path is provided to the target structure from the skin region. When the present systems are used in combination with bone fixation devices, the present systems can provide a suitable path from the skin surface to a bony target structure, such as vertebrae, of the patient. Kits containing the present systems and methods of using such systems are also described.

### FIELD OF THE INVENTION

The present invention relates generally to medical devices and methods. More specifically, the present invention relates to medical devices that provide access to a target site of a patient for medical procedures, as well as methods of using such devices, and kits or packages containing such devices.

### BACKGROUND

In order to perform a surgical procedure on a target structure of a patient, such as when accessing vertebral regions in a patient's body, it is often necessary or at least desirable to dilate the tissue to provide access to the target structure. A common method of dilating tissue in connection with such procedures employs multiple separate cannula dilators. Typically, a set of dilators having different diameters will be used. For example, a first dilator of a relatively small outer diameter will be inserted through the patient's skin toward a target structure. A second cannula having an inner diameter substantially equal to the outer diameter of the first dilator will be placed over the first dilator. A third cannula having an inner diameter substantially equal to the outer diameter of the second cannula will be placed over the second cannula. This stepwise dilation of tissue proceeds until an adequate access path is provided to the target structure.

This method however requires substantial amounts of time in terms of requiring serial steps of dilating the tissue and poses significant risks in terms of increased possibilities that the dilators may be misplaced, unorganized, become contaminated, and the like.

Thus, there remains a need for tissue dilation systems that are easy to use and do not substantially damage tissue that has been dilated.

### SUMMARY

Tissue dilation systems are described which effectively dilate body tissue or tissues of a patient to provide access to a target structure in a patient so that a physician can perform a surgical or medical procedure of the target structure. The present systems employ an expandable member that can be inserted into body tissue in an un-fully expanded configuration, and that can dilate the surrounding tissue by being deployed into a fully expanded configuration. The tissue is maintained in a dilated configuration by placement of a sleeve over the expanded member in the patient's body. Removal of the expanded member from the sleeve provides access for a physician to the target structure. The present systems can be used to dilate tissue in connection with medical procedures involving bones, including vertebrae, the heart, kidneys, lungs, liver, stomach, and other organ and body tissues.

In one embodiment, a tissue dilation system comprises a tissue penetrating member which includes an expandable body portion, and a sleeve including a lumen dimensioned to accommodate the expandable body portion. The expandable body portion is deployable from a first configuration suitable for insertion through a skin region, such as through the skin and fascia, of a patient toward a target structure located in the patient, to a fully expanded second configuration effective in dilating tissue located around the body portion and between the skin region and the target structure when the tissue penetrating member is located in the patient. The sleeve is configured to be placed over the expanded body portion in a fully expanded configuration, and provides access to the target structure from the skin region.

In one specific embodiment, the tissue penetrating member of the foregoing system further includes a substantially rigid wire extending through the expandable body portion to the distal end of the tissue penetrating member. The expandable body portion comprises an inflatable balloon circumscribing a portion of the length of the wire. The inflatable balloon can circumscribe a minor portion, a major portion, or the entire length of the wire. The sleeve has a length effective in providing an access path from the skin region to a bony target surface, such as a vertebra of the patient.

A method of providing access to a target structure in a patient, comprises inserting an expandable body portion of a tissue penetrating member in a first configuration through a skin region into tissue of a patient; deploying the expandable body portion into a fully expanded second configuration to dilate the tissue surrounding the body portion; placing a sleeve over the expanded body portion in the tissue of the patient to provide access to the target structure from the skin region; and removing the tissue penetrating member from the sleeve. The tissue penetrating member can be removed in either an expanded configuration, including fully and partially expanded configurations, or an unexpanded configuration. The body portion can be expanded by inflating the body portion, or other wise mechanically urging the body portion into an expanded configuration.

Packages, such as kits and the like, may comprise one or more of the tissue penetrating members and/or sleeves in a variety of configurations, as described herein.

Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention. Additional advantages and aspects of the present invention are apparent in the following detailed description, drawings, and claims.

### BRIEF DESCRIPTION OF THE FIGURES

FIG. 1 is an illustration of an expandable tissue penetrating member and sleeve of a tissue dilation system in accordance with the disclosure herein.

FIG. 1A is an illustration of the sleeve being placed partially over the expanded tissue penetrating member of FIG. 1.

FIG. 2 is an illustration of the sleeve placed over substantially all of the expanded tissue penetrating member of FIG. 1.

FIG. 3 is an illustration of an expandable tissue penetrating member in an unexpanded configuration.

FIG. 3A is a distal end view along line 3A-3A of FIG. 3.

FIG. 3B is a cross-section view along line 3B-3B of FIG. 3.

FIG. 3C is a cross-section view similar to FIG. 3B with another lumen configuration.

FIG. 3D is a cross-section view similar to FIG. 3C with another lumen configuration.

FIG. 4 is an illustration of the tissue penetrating member of FIG. 3 in an expanded configuration.

FIG. 4A is a distal end view along line 4A-4A of FIG. 4.

FIG. 5 is a cross-section view along line 5-5 of FIG. 4.

FIG. 6 is a cross-section view along line 6-6 of FIG. 3.

FIG. 7 is a cross-section view of a expandable tissue penetrating member in an expanded configuration with a sleeve disposed thereon.

FIG. 8 is an illustration of the tissue dilation system of FIG. 1 and further including a fluid delivery device effective in expanding the tissue penetrating member, and a sleeve partially advanced over an expandable body portion of the tissue penetrating member.

FIG. 9 is an illustration of an expandable tissue penetrating member being inserted through the skin of a patient toward a bony target surface in an unexpanded first configuration.

FIG. 10 is an illustration of the expandable tissue penetrating member of FIG. 9 in an expanded configuration with a sleeve proximally located to the tissue penetrating member.

FIG. 11 is an illustration of the expandable tissue penetrating member of FIG. 10 with the sleeve located around the expanded tissue penetrating member.

FIG. 12 is an illustration of the sleeve providing access to a bony target surface from the skin of a patient, with the tissue penetrating member removed therefrom.

FIG. 13 is an illustration of one method of using the present tissue dilation systems.

FIG. 14 is an illustration of a package or a kit containing one expandable tissue penetrating member and a plurality of sleeves having different lengths.

FIG. 15 is an illustration of a package or a kit containing a three expandable tissue penetrating members having different maximum diameters, and three sleeves having lumen diameters corresponding the maximum diameters of the tissue penetrating members.

FIG. 16 is an illustration of a distal end portion of an expandable tissue penetrating member having a flared distal end.

FIG. 17 is an illustration of a distal end portion of an expandable tissue penetrating member having a second flared distal end configuration.

FIG. 18 is an illustration of a distal end portion of an expandable tissue penetrating member having flared distal end using a balloon having an inverted distal tip configuration.

FIG. 18A is an illustration of a tissue penetrating member similar to FIG. 18 but having a distal end diameter equal to the diameter of a central portion of the tissue penetrating member.

FIG. 19 is an illustration of a tissue penetrating member in which the distal end of the member is located in a target surface.

FIG. 20 is an illustration of an expandable tissue penetrating member in an unexpanded configuration and including a plurality of deformable struts.

FIG. 21 is an illustration of the tissue penetrating member of FIG. 20 in an expanded configuration.

FIG. 22 is an illustration of an expandable tissue penetrating member having a coiled element that selectively expands the distal end portion of the member.

FIG. 23 is an illustration of a sleeve having four longitudinal slits effective in allowing the sleeve to expand at the sleeve's distal end.

FIG. 24 is an illustration of a sleeve having one slit, similar to FIG. 23.

FIG. 25 is an illustration of a collapsible sleeve of the present tissue dilation systems.

FIG. 26 is an illustration of a sleeve having one slit effective in permitting the distal end of the sleeve to be coiled within itself.

FIG. 27 is an illustration of a tissue penetrating member having a dispensing port at the distal tip.

FIG. 28 is a cross-section view of a tissue penetrating member in an expanded state and including a central structural element having a longitudinal groove.

FIG. 29 is a cross-section view of the tissue penetrating member of FIG. 28 in an unexpanded configuration.

FIG. 30 is a cross-section view of a tissue penetrating member including two sidewalls.

### DETAILED DESCRIPTION

A tissue dilation system in accordance with the disclosure herein comprises a tissue penetrating member and a sleeve that is structured, such as sized and shaped, to be placed over the tissue penetrating member. The tissue penetrating member of the present systems is structured to be inserted through the skin of a patient toward a target surface. In the illustrated embodiments described herein, the tissue penetrating member is used in conjunction with medical procedures of bony structures, such as vertebrae of the patient. However, the present systems can be effectively used to dilate tissue and provide access to any target structure from a skin surface of the patient. For example, the present systems can be used to dilate tissue and provide access to the heart, kidneys, liver, stomach, and other organs of the body.

When the tissue penetrating member is placed at a desired position in the patient's body, a portion of the tissue penetrating member is expanded to dilate the tissue surrounding the tissue penetrating member. The sleeve is then placed around the expanded portion of the tissue penetrating member in the patient's body. After removal of the tissue penetrating member from the sleeve, the sleeve effectively provides an access path to the target structure of the patient. Using this access path, a physician can operate or perform a surgical or medical procedure on the target structure. The present systems are easy to operate, provide relatively quick access to desired target structures relative to existing methods, and provide little damage to tissue in proximity to the target structure. The present systems are effective in dilating tissue without cutting the tissue.

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, front, backward, forward, distal and proximal are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims.

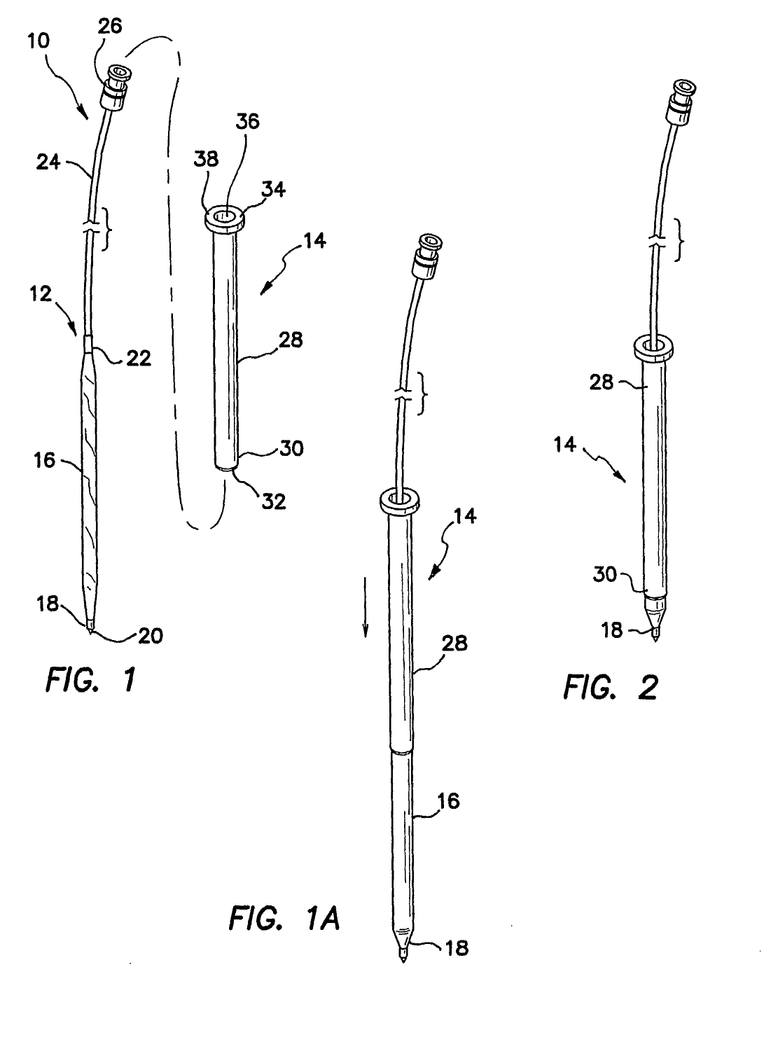

As shown in FIG. 1, a tissue dilation system 10 comprises a tissue penetrating member 12 and a sleeve or sheath 14. The tissue penetrating member 12 includes or comprises an expandable body portion 16. The tissue penetrating member 12 has a distal end region 18 with a distal end element 20, and a proximal end region 22, as shown in FIG. 1. Extending from the proximal end region 22 of the tissue penetrating member 12 is tubing 24 which is connected to a connector 26. The tissue penetrating member 12 may be understood to be a trocar. The connector 26 may be understood to be a luer connector. As understood by persons of ordinary skill in the art, the configuration of the tissue penetrating member 12 and the connector 26 can vary, and are not limited to a trocar or luer connectors.

The sleeve 14 is illustrated as including a body 28 and having a distal end region 30 and a proximal end 34. The distal end region 30 includes a chamfered distal end edge 32. The proximal end region 34 includes an outwardly extending flange element 38. The body 28 of the sleeve 14 includes a lumen 36 extending the length of the body. The sleeve may be understood to be a cannula or a dilator. The inner diameter of the sleeve 14 (i.e., the diameter of the lumen) is substantially equal to the outer diameter of the body portion 16 in a fully expanded configuration. For example, the lumen diameter may be precisely equal to the maximum diameter of the body portion 16, or the lumen diameter may be slightly greater than the maximum diameter of the body portion 16 in the fully expanded configuration. The sleeve 14 is dimensioned to be slidably placed over the fully expanded body portion 16 without substantially engaging or interfering with the body portion 16.

As shown in FIG. 1A, the sleeve 14 is slid or placed over the body portion 16 when the body portion is in a fully expanded configuration. As described herein, the sleeve 14 is typically slid over the body portion 16 when the body portion 16 is located in a body of a patient (i.e., a human or animal patient) and has been deployed into an expanded configuration. As shown in FIG. 2, the sleeve 14 is distally advanced relative to the body portion 16 so that the distal end region 30 of the sleeve 14 is in proximity, preferably adjacent, to the distal end region 18 of the tissue penetrating member 12. It can be appreciated that the sleeve can provide additional tissue dilation due to the thickness of the sleeve sidewall.

Thus, the present tissue dilation system 10 comprises a tissue penetrating member 12, which includes an expandable body portion 16, and a sleeve 14, which includes a lumen 36 that is dimensioned to accommodate the body portion of the tissue penetrating member in its fully expanded configuration. As discussed herein, the expandable body portion 16 is deployable from a first configuration, such as an unexpanded configuration, to a fully expanded second configuration. In the first configuration, the expandable body portion 16 is suitable for insertion through a skin region of a patient toward a target structure in the patient. As used herein, a skin region is understood refers to a portion of skin of a patient, and can include the fascia underlying the skin. The tissue penetrating member 12 can be inserted through an incision in the skin region, such as an incision formed in the skin and underlying fascia, or the tissue penetrating member 12 can be directly inserted through an uncut skin region. In the second configuration, the expandable body portion 16 dilates the tissue located around the body portion. When the tissue penetrating member 12 is removed from the sleeve 14, access from the skin region to the target structure can be obtained.

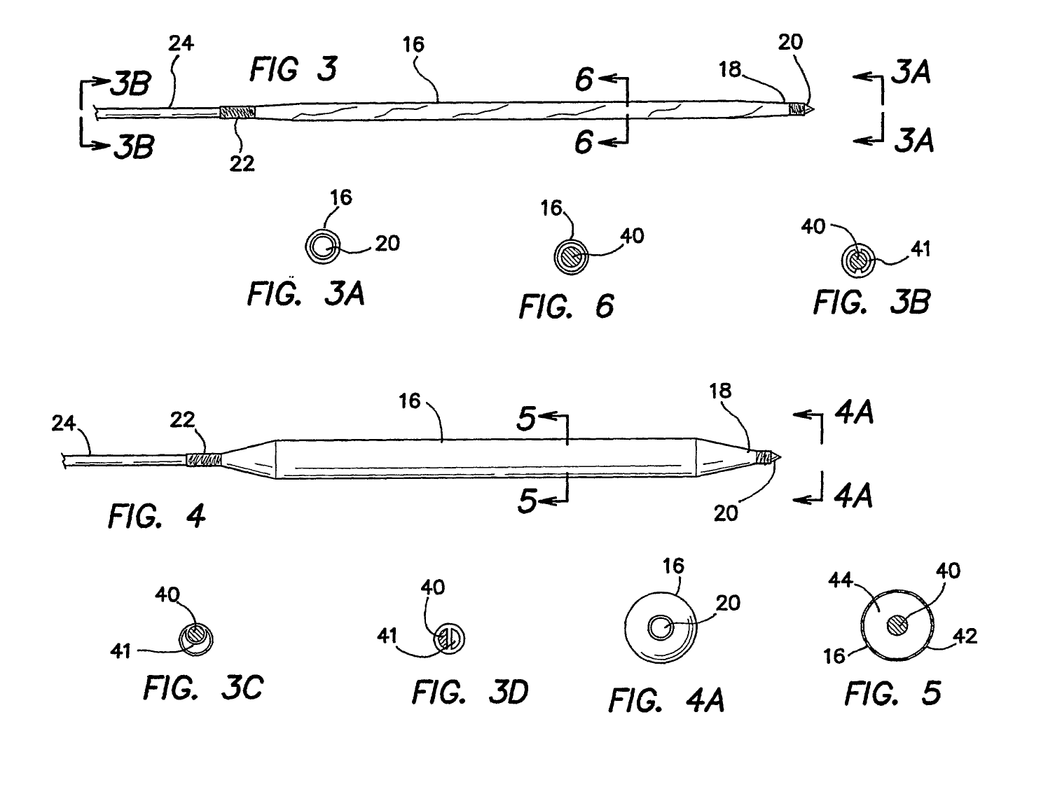

The expandable body portion 16 of the tissue penetrating member 12 is illustrated in an unexpanded configuration in FIG. 3. In reference to the disclosure herein, the unexpanded configuration may be understood to be a first configuration. In this first configuration, the tissue penetrating member 12 is suitable for insertion through tissue in a patient. The maximum diameter of the expandable body portion 16 in the first configuration is substantially similar to the maximum diameter of the tubing 24 or the maximum diameter of the distal end tip 20 shown in FIG. 3. Thus, the tissue penetrating member 12 can be readily inserted through bodily tissue without substantially damaging the tissue through which it is inserted. The relative diameters of the body portion 16 and the distal end tip 20 are more clearly seen in FIG. 3A. Although the first configuration is illustrated as being an unexpanded configuration, the first configuration may also be understood to encompass partially expanded configurations, such as configurations in which the maximum diameter of the body portion 16 is greater than in an unexpanded configuration but less than the maximum diameter in a fully expanded configuration, described herein.

As shown in FIG. 4, the expandable body portion 16 can be deployed from the first configuration (FIG. 3) to a second fully expanded configuration. In this second configuration, the maximum diameter of the body portion 16 is substantially greater than the maximum diameter of the distal end tip 20 or the tubing 24, as seen in FIG. 4A. For example, the maximum diameter of the body portion in the second configuration may be at least about two times greater than the maximum diameter of the distal end tip 20. In certain embodiments, the maximum diameter of the body portion 16 in the second configuration may be from about two times greater to about fifty times greater than the maximum diameter of the body portion in the first configuration. In one embodiment, the maximum diameter of the body portion is about 10 times greater than the maximum diameter of the distal end tip 20 or of the maximum diameter of the body portion in the first configuration. The maximum diameter of the body portion 16 in the second configuration is effective in dilating the tissue surrounding the body portion when it is located in the body of a patient. The maximum diameter should be sufficient to provide physical access for a physician to perform a surgical or other medical procedure on the target structure. However, the diameter should not be excessively large to cause unwanted damage to the surrounding tissue and result in potential injury to the patient.

As shown in FIGS. 5 and 6, the tissue penetrating member 12 may further include or comprise a structural element 40 located within the expandable body portion 16. In the illustrated embodiment, the structural element 40 is a substantially rigid wire extending from the proximal end region 22 to the distal end region 18. Furthermore, in reference to the drawings, the distal end tip 20 of the tissue penetrating member 12 corresponds to the distal end tip of the structural element 40. In the embodiments shown in FIGS. 1-6, the expandable body portion is an inflatable balloon or balloon-like device. Thus, the structural element 40 provides structure to the tissue penetrating member 12 and more specifically to the body portion 16 to facilitate insertion of the tissue penetrating member 12 through a patient's body tissue. However, as described herein, other embodiments may not require a structural element as illustrated in FIGS. 1-6.

Thus, as shown in FIGS. 1-6, the expandable body portion 16 may be understood to comprise an inflatable sheet 42 encompassing the structural element 40. In one embodiment, the inflatable sheet 42 is a balloon. The inflatable sheet 42 is sealed around the structural element 40 near the proximal end region 22 and the distal end region 18 of the tissue penetrating member. The cavity 44 defined by the inflatable sheet 42 is in fluid communication with tubing 24. Accordingly, fluid, such as gas or liquid, can be delivered through the tubing 24 and into the cavity 44 to deploy the body portion 16 or sheet 42 into a fully expanded configuration.

Certain embodiments of the present tissue penetrating members may comprise an inflatable balloon, as described herein. Some embodiments may comprise a non-compliant balloon, other embodiments may comprise a semi-compliant balloon, and other embodiments may comprise a fully compliant balloon. Balloon compliance describes the degree to which the balloon will inflate as pressure inside the balloon increases. These types of balloons of other medical devices are understood by persons of ordinary skill in the art. Thus, the present systems may comprise a balloon having a single maximum diameter when inflated. Or, the present systems may comprise a balloon that has a variable maximum diameter that is related to the amount of pressure used to inflate the balloon. For example, one system may comprise a balloon that when fully expanded has a maximum diameter of about 14 mm. Another system may comprise a balloon that has a maximum diameter of about 14 mm when a first amount of pressure is used to inflate the balloon, and a second maximum diameter of about 18 mm when a second amount of pressure is applied to inflate the balloon. In reference to the embodiments illustrated in FIGS. 1-6, the maximum diameter remains substantially constant along the length of the balloon.

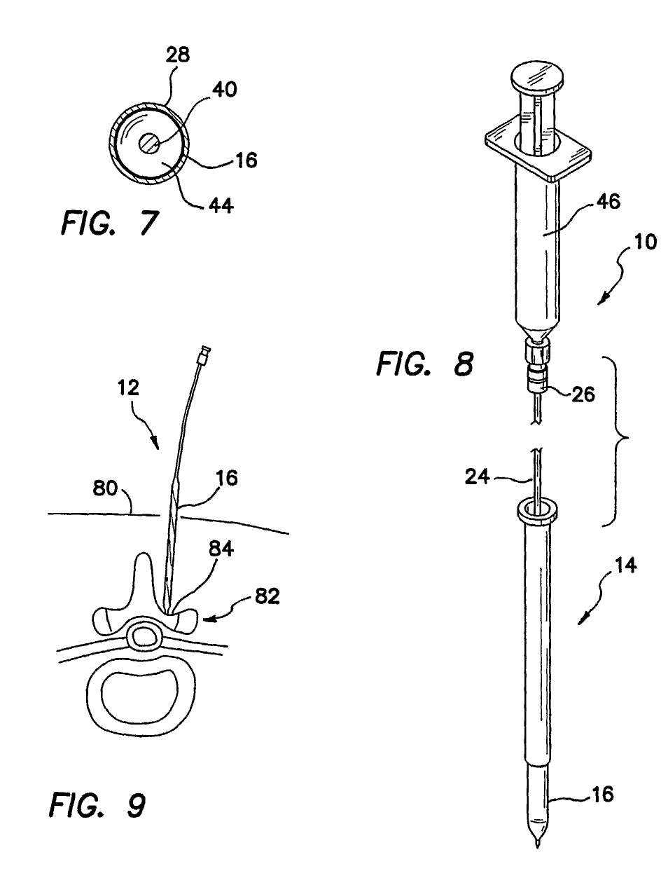

FIG. 7 illustrates a cross-section view of the body 28 of the sleeve 14 located over the expandable body portion 16 in a fully expanded configuration. It can be observed that the lumen diameter of the body 28 is substantially equal to the outer diameter of the expanded body portion 16.

As shown in FIG. 8, the present tissue dilation systems may further comprise a fluid delivery device 46 coupled to the tissue penetrating member 12. As illustrated, the fluid delivery device 46 is coupled to tissue penetrating member 12 by tubing 24, which can include a conventional luer connector. The tubing, or a portion of the tubing, may comprise a portion of the structural element 40. Thus, the tubing may have two or more lumens, one of the lumens containing a portion of the structural element 40. The tubing may be dual lumen tubing, coaxial lumen tubing, and the like. One example of the tubing with a centrally disposed guide wire is shown in FIG. 3B. A similar or identical view may also be seen when the connector, such as a luer connector, is viewed from the proximal end of the connector towards the distal end of the connector. Other examples of tubing are shown in FIGS. 3C and 3D. In reference to these figures, the structural element 40 is located in the tubing 24 and is adjacent a fluid delivery lumen 41. Other embodiments of the tubing may comprise three or more lumens, as desired. One or more lumens may be used for drug delivery to the patient. As shown in FIG. 8, the fluid delivery device 46 can be a syringe. In certain embodiments, the fluid delivery device may include a pressure gauge facilitating delivery of the proper amount of fluid to the expandable body portion 16. One example of a suitable fluid delivery device is a balloon angioplasty inflator or an indeflator (Guidant Corp.). In other embodiments, any suitable pumping device or device which can deliver fluid at a predetermined pressure can be coupled to the tissue penetrating member to achieve the desired tissue dilation.

Certain embodiments of the present systems include a sleeve 16 that has a body 28 having a length substantially equal to the distance from the skin region to the target structure. In other words, the sleeve 16 includes a body 28 that extends substantially from the target structure to the skin surface of the patient. This is in contrast to cardiovascular devices, such as stents, that may be inserted in a blood vessel over an angioplasty balloon. Such cardiovascular devices do not have a length that extends from the target surface to the skin region or skin surface of a patient. In addition, or alternatively, the sleeves of the present systems may be longer than the skin to target structure distance.

The sleeve 14 of the present systems is formed of a substantially rigid material, such as hardened plastic materials and the like. By using a substantially rigid material, the sleeve 14 can maintain the surrounding tissue in a dilated configuration. In other words, the surrounding tissue does not noticeably constrict the sleeve and thereby reduce access to the target structure. In addition, the sleeve 14 is substantially smooth. For example, the sleeve 14 does not include surface irregularities that may damage the surrounding body tissue or interfere with the movement between the sleeve 14 and the tissue penetrating member 12. In certain embodiments, the inner wall of the lumen 36 or the outer surface of the body portion 16, or both may include a lubricant to reduce potential negative interactions between the sleeve and the tissue penetrating member.

Thus, in at least one specific embodiment, such as an embodiment that is effective to provide tissue dilation of tissue in proximity to a bony surface, a tissue dilation system comprises an tissue penetrating member and a sleeve, as described herein. The tissue penetrating member includes a substantially rigid wire extending through the expandable body portion to the distal end of the tissue penetrating member, and the expandable body portion comprises an inflatable balloon circumscribing a portion of the length of the wire. The sleeve of the system has a length effective in providing an access path from the skin region to a bony target surface, such as a vertebra of the patient.

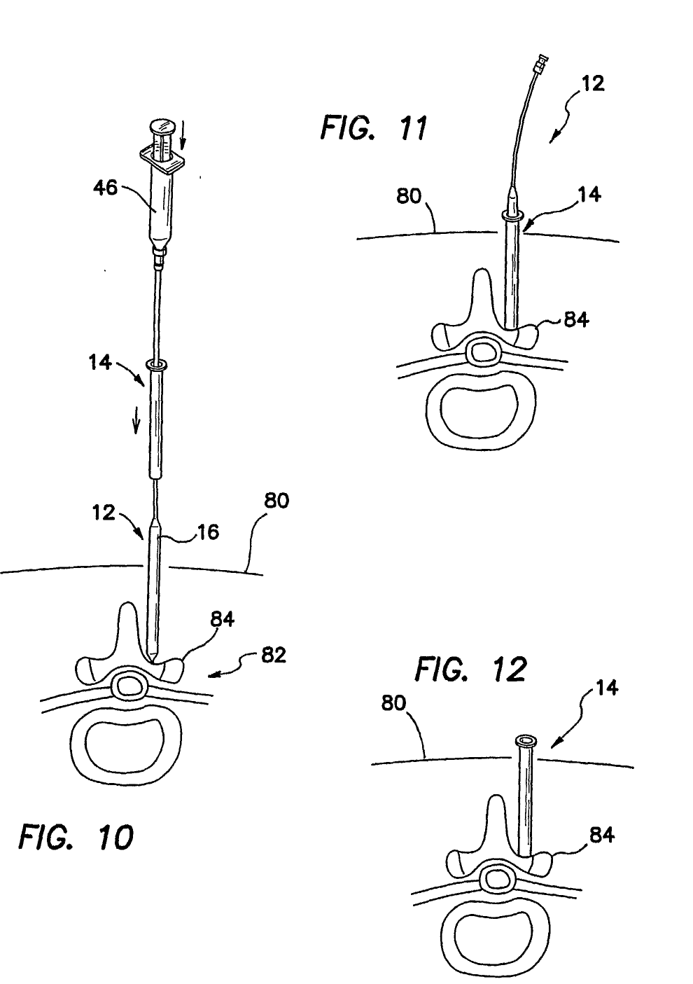

An example of such an embodiment is illustrated in use in FIGS. 9-12. As shown in FIG. 9, the expandable body portion 16 of the tissue penetrating member 12 has been inserted through the skin or a skin region 80 of a patient. The expandable body portion 16 has been inserted in a first configuration, as described herein, and has been inserted towards a target structure 82, such as a vertebra 84. The expandable body portion 16 has been deployed into an expanded configuration in FIG. 10 by pressure delivered by the fluid delivery device 46. The sleeve 14 is being placed over the expanded body portion 16. In FIG. 11, the sleeve 14 has been placed over the expanded body portion 16. The distal end of the sleeve 14 is placed substantially adjacent to the vertebra 84. As shown in FIG. 12, the tissue penetrating member has been removed from the sleeve 14. The sleeve 14 thereby provides access to the vertebra 84 for a surgical or medical procedure, such as for the placement of a bone fixation device. As shown in FIG. 12, the length of the sleeve 14 is substantially equal to the distance between the skin surface 80 and the target structure, such as vertebra 84.

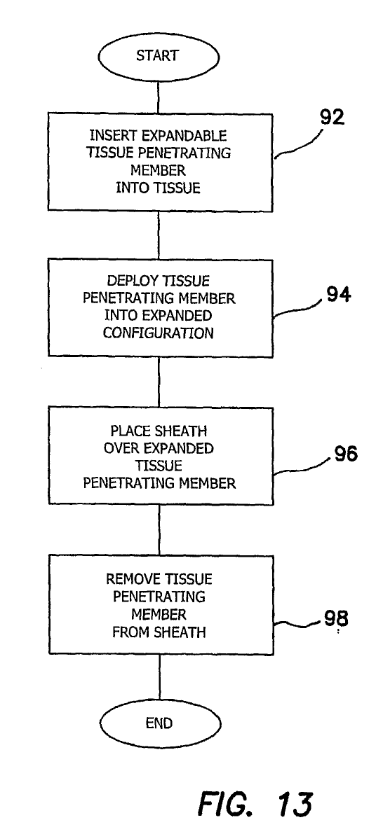

A method of using the present systems is illustrated in the flow chart of FIG. 13. The method shown in FIG. 13 comprises a step 92 of inserting an expandable body portion of a tissue penetrating member in a first configuration through a skin region and into tissue of a patient. After the body portion is inserted through the skin region, the method comprises a step 94 of deploying the expandable body portion into a fully expanded second configuration. As discussed herein, the expansion of the body portion is effective to dilate the tissue surrounding the body portion. Once the tissue has been dilated, the method comprises a step 96 of placing a sleeve or sheath over the expanded body portion in the tissue of the patient. Thus, the sleeve provides access to the target structure from the skin region. With the sleeve substantially in place, the method comprises a step 98 of removing the tissue penetrating member from the sleeve.

Optionally, the foregoing method may comprise a step of making an incision in the skin region of the patient before inserting the tissue penetrating member. The incision can be made using any conventional technique. In certain situations, the skin and fascia will be cut to provide direct access to the underlying muscle and/or tissue.

As discussed herein, the present systems may comprise an expandable body portion that is inflatable by administration of a fluid, such as a gas or liquid. Thus, the foregoing method may comprise a step of directing fluid into the inflatable body portion to deploy the body portion into the expanded configuration. As indicated herein, in such situations, the body portion may comprise an inflatable balloon.

As discussed herein, the tissue penetrating member can be removed from the sleeve in a fully expanded configuration. However, the tissue penetrating member can also be urged into an unexpanded configuration before removal from the sleeve. When the body portion comprises an inflatable balloon, the method may comprise deflating the balloon before removal from the sleeve.

The present invention also relates to packages, containers, or kits comprising the present systems. For purposes of convenience, the foregoing will be collectively referred to as packages. The packages may also include other elements that may be useful in using the present tissue dilation systems. For example, the packages may include instructions for using the systems. The instructions may include a description of the methods described herein. Or, the packages may comprise other surgical or medical items, including a fluid delivery device(s), such as an indeflator or syringe, as described herein. For example, the packages may comprise one or more bone fixation devices, including the devices disclosed in U.S. Pat. Nos. 6,685,706; 6,648,890; 6,632,224; 6,511,481; and 6,348,053, and U.S. Pat. Pub. Nos. 20050033289; 20040260297; 20040260289; 20040199165; 20040199162; 20040181222; 20040138665; 20040127906; 20040106925; 20040010257; 20030097132; 20030069582; 20020143335; 20020143334; 20020143333; 20010049530; and 20010049529.

As shown in FIG. 14, a package 50 may comprise one tissue penetrating member 12, and a plurality of sleeves 14. Although three sleeves are illustrated, the package may comprise two sleeves or four or more sleeves. In the package 50, the sleeves have different lengths. For example, the package may only comprise one sleeve of any given length. In other packages, there may be two or more sleeves of the same length as well. By providing sleeves of different lengths, it is possible to provide a single package suitable for dilating tissue at a variety of different target sites of a patient. Thus, the package 50 may comprise the system illustrated in FIG. 1, for example, and at least one additional sleeve having a different length than the other sleeve.

Another package 52 is shown in FIG. 15. The package 52 comprises a plurality of the present tissue dilation systems. For example, the package 52 comprises three different tissue penetrating members 12 and three different sleeves 14. Each of the tissue penetrating members 12 have a different maximum diameter in a fully expanded configuration, and each of the sleeves 14 have different lumen diameters. The lumen diameters correspond to the respective maximum diameters of the tissue penetrating members.

Another package, or article, may comprise individually packaged tissue penetrating members and a plurality of packages of sleeves having different lengths, each sleeve being provided in a single package. Such packages containing the sleeves or tissue penetrating members may be sealed to maintain the devices in sterile conditions. These tissue penetrating members may have one or more markings indicating a desirable length of sleeve to be used in combination with the tissue penetrating member. Thus, such packages or articles may be effective in permitting a user to select a single tissue penetrating member, and a single sleeve of a desired length from among a plurality of other sleeves.

In view of the disclosure herein, it can be understood that the present tissue dilation systems comprise a tissue penetrating member which has an expandable portion, such as an inflatable portion, that can dilate tissue in a patient's body once that portion is placed in the tissue, and a sleeve to retain the tissue in a dilated configuration. It is desirable that the diameter of the access path through the dilated tissue does not substantially decrease towards the target structure. In other words, it is desirable that the diameter of the access path remain substantially constant from the skin surface to the target structure, or that the diameter of the access path increases near the target structure. One object of the present systems is to reduce the distance from the target structure to the maximum diameter of the access path or the maximum diameter of the tissue penetrating member. Another object may be to increase or maximize the working area near the target structure by providing a greater dilation at the distal end of the tissue penetrating member and/or sleeve relative to more proximal regions of the tissue penetrating member and/or sleeve. Thus, several additional embodiments of the present tissue penetrating members and sleeves are described herein.

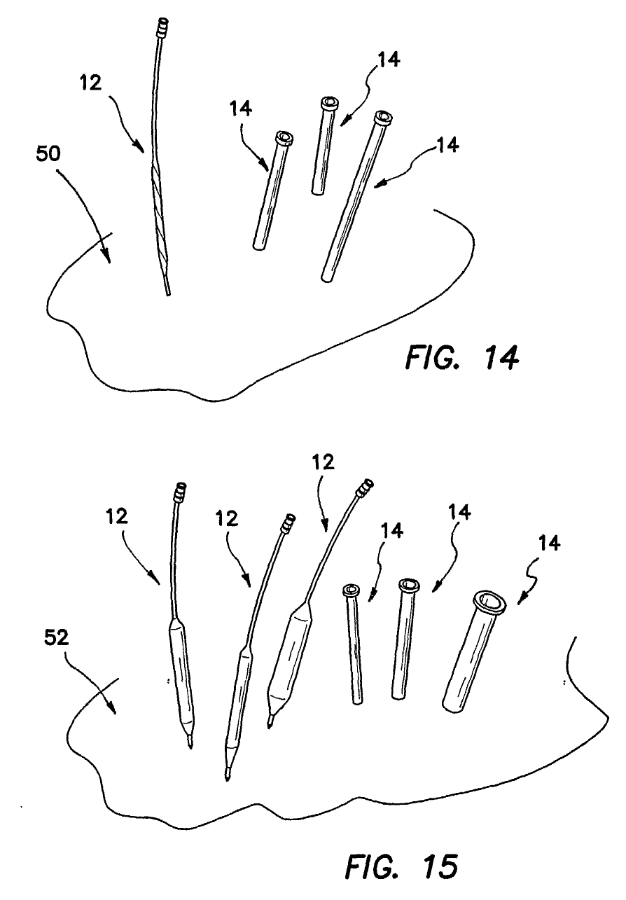

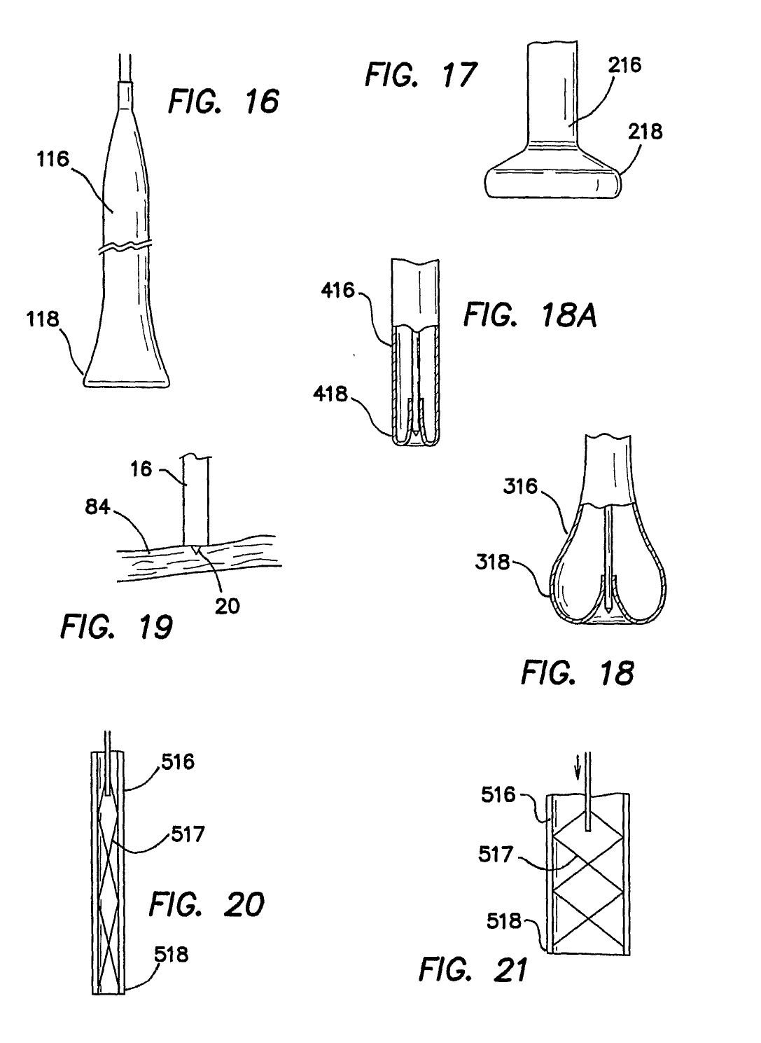

In short, embodiments such as those illustrated in FIGS. 18A and 19, are effective in minimizing the distance between the target structure and the region of the tissue penetrating member having a maximum diameter. Embodiments, such as those illustrated in FIGS. 16, 17, 18, and 22, are effective in increasing the working area near the target structure. In addition, embodiments, such as those illustrated in FIGS. 16, 17, 18, and 22 may be effective in achieving both objects of minimizing tip distance and increasing working area.

The present tissue penetrating members may comprise an expandable body portion that have varying maximum diameters along the length of the expandable body portion. For example, the tissue penetrating members may comprise a varying diameter balloon that has a diameter that varies along the length of the balloon. One example of a varying diameter expandable body portion is illustrated in FIG. 16. The expandable body portion 116 comprises a flared distal end 118. More specifically, the expandable body portion 116 has a diameter that increases distally along the length of the body portion until the distal end of the body member. Another example is illustrated in FIG. 17. In this embodiment, the rate of change in diameter from the expandable body portion 216 to the distal end 218 is greater than that shown in FIG. 16. In addition, the distal end 218 includes a region of substantially constant maximum diameter that may provide improved access to the target structure.

FIG. 18 depicts a flared expandable body portion 316 having a flared distal end 318. The expandable body portion 316 has a distal end in the form of an inverted balloon, similar to inverted balloons used in angioplasty procedures. Another embodiment using a balloon with an inverted distal end region is shown in FIG. 18A. In this embodiment, the maximum diameter of the body portion 416 is substantially constant along the length of the body portion to the distal end 418.

Balloons having flared or enlarged distal end regions may be formed of different materials along the length of the balloon. For example, a region of the balloon proximal to the enlarged region may comprise a material having a single maximum expansion size, and the enlarged region may comprise a material having a dual maximum expansion size. Thus, upon application of a given amount of pressure, such a balloon can expand to the maximum size of the more proximal region, and upon further application of an additional amount of pressure, the distal end region can expand to the second maximum expansion size without changing the maximum expansion size of the more proximal region of the balloon.

An embodiment of a tissue penetrating member having a non-tapered expandable body portion is shown in FIG. 19. In this embodiment, the distal end of the expandable body portion 16 is substantially adjacent the distal end tip 20 of the tissue penetrating member. This embodiment can be understood to be the same embodiment as that described in FIGS. 1-6. To reduce the distance between the target structure surface, such as bony surface 84, the distal end tip 20 is inserted into the bone so that the distal end of the expandable body portion 16 abuts the bony surface 84.

In addition to using a fluid to expand the expandable body portion of the present tissue penetrating members, other embodiments may use different mechanisms for deploying the body portion from a first configuration to a fully expanded second configuration. For example, as shown in FIGS. 20 and 21, the expandable body portion 516 can comprise a plurality of struts 517 that extend along the length of the body portion to the distal end 518. The struts may lie adjacent the inner sidewall of the expandable body portion. The struts are deformable so that the body portion can be moved between a first unexpanded configuration (FIG. 20) and a fully expanded second configuration (FIG. 21). In certain embodiments, the struts are biased towards the first or second configuration. For example, struts that are biased towards the first configuration may require positive pressure applied toward the distal end of the body portion to expand the body portion. Alternatively, struts that are biased towards the expanded configuration can be “pulled” into an unexpanded configuration for insertion of the body portion into the body tissue, and then released into the fully expanded configuration.

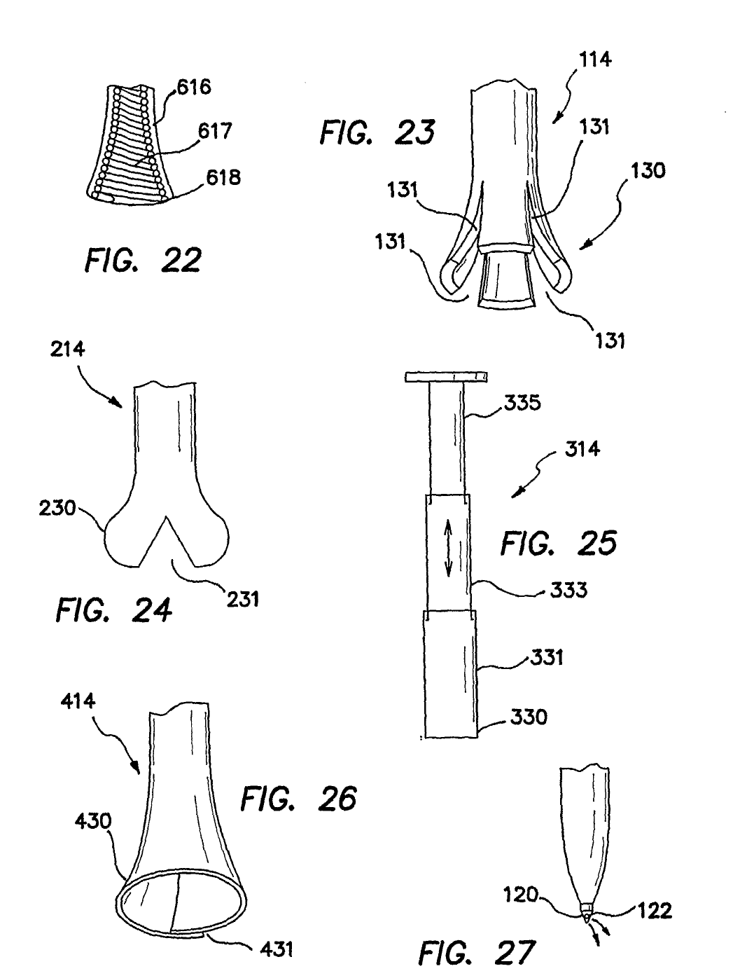

Another embodiment of the tissue penetrating member may include a coil element 617 shown in the expandable body portion 616 in FIG. 22. The coil may wrap around a central longitudinal axis of the tissue penetrating member or body portion. Active rotation of the coil element 617 when the distal end 618 of the body portion 616 is adjacent a target structure can cause the coil to expand the distal end 618 of the body portion to obtain the desired flared configuration. A similar concept of using a coil element on the inner surface of a sleeve can be used to cause the sleeve to flare at its distal end.

The present systems may also comprise sleeves that can take advantage of the flared or altered dilation obtained with the tissue penetrating members. For example, as shown in FIG. 23, a sleeve 114 may have a distal end 130 with four slits 131 extending longitudinally from the distal end 130. The slits permit the distal end to flare outwardly to accommodate flared expandable body portions. As shown in FIG. 24, a sleeve 214 can comprise a single slit 231. As shown in FIG. 26, a sleeve 414 may comprise a slit 431 which permits one end of the distal end 430 to be inserted into the other end, as shown.

Sleeves having one or more slits may also include a material effective in reducing movement of dilated tissue into the interior of the sleeve. Such materials may be expandable and or stretchable. The materials may be provided as a coating over the sleeve, or may be provided as a layer extending between sleeve portions on either side of a slit. For example, the materials may be provided as a biocompatible elastic coating over the distal end of the sleeve, or may be provided as a webbing-like structure coupled to each side of the sleeve portion defining a slit. The material may be porous or non-porous.

Another embodiment of the present systems comprises a collapsible sleeve 314, as shown in FIG. 25. The sleeve 314 has a distal end 330. The sleeve 314 may also be understood to include a distal region 331, a central region 333, and a proximal region 335. In this embodiment, different lengths of the sleeve can be obtained by moving the proximal region into the central region, and/or moving the central region into the distal region. Although the three regions are illustrated as having substantially equal lengths, other embodiments can have more or fewer regions, and regions of different lengths.

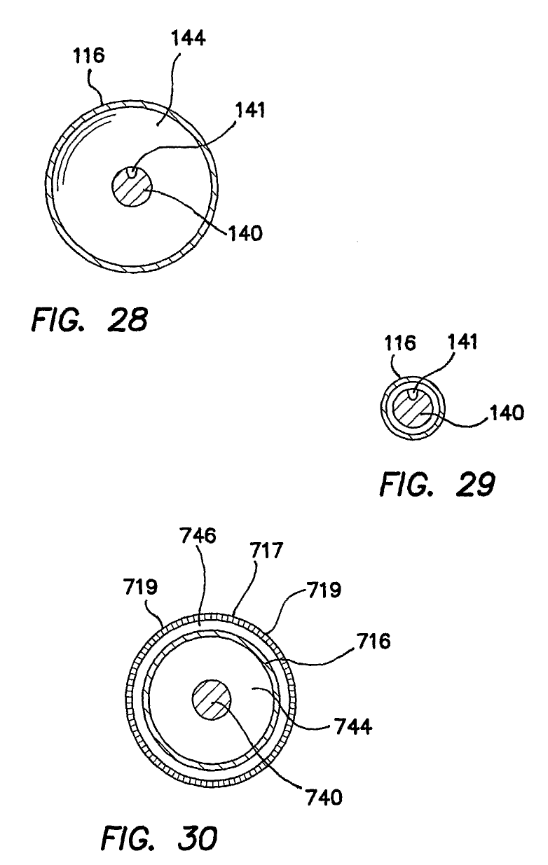

The present systems may also be used to deliver a medication or pharmaceutical composition to the surrounding tissue. For example, the tissue penetrating member and/or sleeve may include one or more ports that can pass a substance to the tissue. As shown in FIG. 27, a dispensing port 122 can be provided at the distal end tip 120. Material may be directed toward the dispensing port through a channel 141 of the structural element 140 shown in FIGS. 28 and 29, for example. For example, the port can be used to deliver muscle relaxants, analgesics, local anesthetics, and temperature adjusting agents, which may help during the procedure and recovery from the procedure.

As shown in FIG. 30, dispensing ports 719 can be provided in a sidewall of the expandable body portion. More specifically, in reference to FIG. 30, the expandable body portion 716 comprises an inflatable balloon. In this embodiment, the balloon is a double walled device. For example, the balloon defines the cavity 744 located around the structural element 740. A second sidewall 717 is provided around the body portion 716. The second sidewall 717 has a plurality of pores 719 located therein. A composition, such as a liquid containing composition, can be delivered into the outer cavity 746 where the composition can pass through the pores 719 and contact the surrounding tissue. For example, the ports or pores can be used to deliver muscle relaxants, analgesics, local anesthetics, and temperature adjusting agents, which may help during the procedure and recovery from the procedure.

The present systems can be made using conventional techniques and materials. Preferably, the materials are suitable for medical purposes. As discussed herein, the sleeves can be extruded or injection molded from plastic materials. The tissue penetrating member can be produced by placing an inflatable sheet around a structural element, such as a wire, and coupling the cavity defined by the inflatable sheet to a tubing lumen, or the tissue penetrating member can be produced by placing a sheet on the exterior of a plurality of struts or a coil element, which impart their own structural features.

Additional embodiments of the present systems may include markings or indicia provided on the expandable body portion to assist in determining the particular distance the sleeve has been positioned in the body. In addition, the sleeve and/or tissue penetrating member can also include other positioning facilitator elements. For example, the sleeve can include a radiopaque material, such as barium sulfate, bismuth compounds, and tungsten metals. The tissue penetrating member can include contrast media. Contrast media may be provided in the fluid used to inflate the balloon of the balloon-embodiments disclosed herein.

All references, articles, patents, applications and publications set forth above are incorporated herein by reference in their entireties.

While this invention has been described with respect to various specific examples and embodiments, it is to be understood that the invention is not limited thereto and that it can be variously practiced with the scope of the following claims. Multiple variations and modifications to the disclosed embodiments will occur, to the extent not mutually exclusive, to those skilled in the art upon consideration of the foregoing description.

What is claimed is:

1. A tissue dilation system, comprising:
a tissue penetrating member including an expandable body portion deployable from a first configuration suitable for insertion through a skin region of a patient toward a target structure located in the patient, to a fully expanded second configuration effective in dilating tissue located around the body portion and between the skin region and the target structure when the tissue penetrating member is located in the patient; and
a sleeve including a lumen dimensioned to accommodate the expandable body of the tissue penetrating member in the fully expanded second configuration, the sleeve providing access to the target structure from the skin region.

2. The system of claim 1, wherein the tissue penetrating member further includes a structural element located within the expandable body portion.

3. The system of claim 2, wherein the expandable body portion comprises an inflatable sheet encompassing the structural element.

4. The system of claim 2, wherein the structural element extends beyond the expandable body portion to define a distal end tip of the tissue penetrating member.

5. The system of claim 1, further comprising a fluid delivery device coupled to the tissue penetrating member and effective in delivering a fluid to the expandable body portion to deploy the body portion from the first configuration to the second fully expanded configuration.

6. The system of claim 6, wherein the fluid delivery device comprises a syringe containing gas or liquid to inflate the expandable body portion.

7. The system of claim 1, wherein the sleeve includes a body having a length substantially equal to the distance from the skin region to the target structure.

8. The system of claim 1, wherein the sleeve includes an outwardly extending flange at the proximal end of the sleeve.

9. The system of claim 1, wherein the sleeve includes a chamfered distal end edge surface.

10. The system of claim 1, wherein the sleeve lumen has an inner diameter substantially equal to a maximum outer diameter of the expandable body portion in the fully expanded second configuration.

11. The system of claim 1, wherein the sleeve is formed of a substantially rigid material effective in maintaining the tissue surrounding the sleeve in dilated configuration.

12. The system of claim 1, wherein the tissue penetrating member further includes a substantially rigid wire extending through the expandable body portion to the distal end of the tissue penetrating member, and the expandable body portion comprises an inflatable balloon circumscribing a portion of the length of the wire.

13. The system of claim 12, wherein the sleeve has a length effective in providing an access path from the skin region to a bony target surface.

14. The system of claim 13, wherein the sleeve has a length effective in providing an access path from the skin region to a vertebra of the patient.

15. A package comprising the system of claim 1, and at least one additional sleeve of a different length than the other sleeve.

16. A package comprising a plurality of the systems of claim 1, wherein each system includes a tissue penetrating member having a different maximum diameter in the second fully expanded configuration.

17. The system of claim 1, wherein a distal end portion of the expandable body portion has a greater maximum diameter in the second fully expanded configuration relative to a more proximal region of the expandable body portion.

18. The system of claim 1, wherein the expandable body portion comprises a balloon having an inverted distal end region.

19. The system of claim 1, wherein the expandable body portion comprises a balloon selected from the group consisting of compliant balloons, semi-compliant balloons, and non-compliant balloons.

20. The system of claim 1, wherein the expandable body portion comprises a plurality of struts effective in deploying the body portion from the first configuration to the fully expanded second configuration.

21. The system of claim 1, wherein the expandable body portion comprises a coil element effective in flaring a distal end of the body portion.

22. The system of claim 1, wherein the sleeve has a distal end including at least one longitudinal slit.

23. The system of claim 1, wherein the sleeve includes a distal region, a central region, and a proximal region, the proximal region being collapsible into the central region, and the central region being collapsible into the distal region.

24. The system of claim 1, wherein the tissue penetrating member includes at least one dispensing port for delivering a substance to tissue surrounding the tissue penetrating member.

25. The system of claim 1, wherein the expandable body portion comprises a double-walled balloon.

26. The system of claim 1 in combination with a bone fixation system.

27. A method of providing access to a target structure in a patient, comprising:
inserting an expandable body portion of a tissue penetrating member in a first configuration through a skin region into tissue of a patient;
deploying the expandable body portion into a fully expanded second configuration to dilate the tissue surrounding the body portion;
placing a sleeve over the expanded body portion in the tissue of the patient to provide access to the target structure from the skin region; and
removing the tissue penetrating member from the sleeve.

28. The method of claim 27, further comprising a step of making an incision in the skin region of the patient before inserting the tissue penetrating member.

29. The method of claim 27, wherein the deploying comprises directing a fluid into an inflatable sheet surrounding a wire of the tissue penetrating member.

30. The method of claim 29, wherein the inflatable sheet is a balloon.

31. The method of claim 27, wherein the placing of the sleeve comprises inserting the sleeve to a bony surface located in the body of the patient.

32. The method of claim 31, wherein the sleeve is inserted to a vertebra of the patient.

33. The method of claim 27, wherein the tissue penetrating member is removed from the sleeve in a fully expanded configuration.

34. The method of claim 27, further comprising a step of urging the expanded body portion to a less expanded configuration before removing the tissue penetrating member from the sleeve.

35. The method of claim 27, wherein the expandable body portion comprises a balloon circumscribing a wire extending the length of the body portion, and the deploying comprises inflating the balloon to dilate tissue surrounding the balloon between the skin region and a vertebra of the patient, and the method further comprises deflating the balloon before removing the tissue penetrating member from the sleeve.
